(19) United States
(12) Patent Application Publication
(10) Pub. No.: US 2005/0137256 A1
(43) Pub. Date: Jun. 23, 2005

(54) Use of arachidonic acid as a method of increasing skeletal muscle mass

(76) Inventor: William C. Llewellyn, Palm Beach Gardens, FL (US)

Correspondence Address: Todd S. Hofmeister;Holland & Knight LLP, 30th Floor, 131 S. Dearborn Street, Chicago, IL, 60603, US

(21) Appl. No.: 11/027,472

(22) Filed: Dec. 30, 2004

Related U.S. Application Data
(63) Continuation-in-part of application No. 10/306,265, filed on Nov. 27, 2002, now Pat. No. 6,841,573.

(51) Int. Cl. A61K 31/202; A61K 31/22

(52) U.S. Cl. 514/546; 514/560; 514/548

(57) ABSTRACT

This invention discloses a method of orally administering arachidonic acid and its derivatives for the purpose of increasing the serum level of the prostaglandin PGF2alpha and subsequently the level of retained skeletal muscle mass.

### CROSS REFERENCE TO RELATED APPLICATIONS

This application is a continuation-in-part of co-pending U.S. utility patent application Ser. No. (U.S. Ser. No.) 10/306,265, filed Nov. 27, 2002.

### BACKGROUND OF THE INVENTION

Maintaining a healthy level of muscle mass can play an important role in sustaining overall good health, with benefits such as increased basal metabolic rate, better disposal of dietary fats and maintenance of lower body fat levels, increased immune system health, and an increase in one's overall vitality and sense of well-being compared to maintaining lower than ideal levels of lean body mass. A number of pathological conditions exist that make it difficult to maintain a normal healthy level of muscle mass, including HIV (human immunodeficiency virus), andropause or hypogonadism (subnormal androgen levels), infection, trauma, burns, and spinal cord injury. Some individuals also fail to gain or to maintain normal lean body mass without definite pathophysiologic reasons. Many treatments are offered that would help an individual in need of such treatment promote the buildup of muscle tissue.

Skeletal muscle mass is increased or maintained in the body through a number of separate and distinct mechanisms. Such mechanisms play a role in the regulation of either skeletal muscle protein synthesis or breakdown, and collectively control the total amount of accrued protein present in the muscle cell. The actions of androgens are among the most visibly tied to the regulation of skeletal muscle mass, as these hormones are collectively responsible for the development and maintenance of male sexual characteristics including external virilization, sexual maturity at puberty, spermatogenesis, sexual behavior/libido and erectile functioning and the support of bone and muscle tissue growth. It is well documented in the prior art that raising the level of androgenic hormones in the body can increase skeletal muscle mass. A number of methods have similarly been developed to increase the level of androgenic hormones in the body, which ultimately can be used to offer the benefits of increased skeletal muscle mass in humans.

In searching for ways to increase androgen levels in the body, the use of androgen precursor hormones have been suggested. U.S. Pat. No. 5,578,588 to Mattern et al. relates a method of using a precursor hormone, namely androstenedione, as a means of increasing testosterone levels. Although the in-vivo conversion of endogenous androstenedione to testosterone had been documented, the use of this compound as an external supplement for producing a stable and effective increase in serum testosterone has never been investigated before, and therefore represents a novel invention. The pharmacokinetics of administering such a precursor is such that hormone concentrations of active hormone (testosterone) peak within 90 minutes, and subsequently decline over a period of three to four hours. This more closely resembles the natural pulsating pattern in which the body releases testosterone, and avoids the prolonged peaks and troughs noted with use of esterified injectable hormone preparations.

Several other methods of using different androgen precursor hormones have also been disclosed, including U.S. Pat. No. 5,880,117 to Patrick Arnold, which relates to a method of using 4-androstenediol as a means of increasing testosterone levels in humans. This in-vivo conversion of 4-androstenediol to testosterone, has been well documented; however, the use of this compound as an effective oral medicament for raising testosterone levels has not been investigated. U.S. Pat. No. 6,391,868 to Arnold similarly relates a method of using 5-alpha-androst-1-en-3-one for increasing levels of the anabolic/androgenic steroid 17-beta-hydroxy-5-alpha-androst-1-en-3-one in humans. The in-vivo bioconversion was known; however, a formal investigation of its oral use to increase serum androgen levels has not been disclosed. U.S. Pat. No. 6,262,436 to Llewellyn further discloses the method of using 5-alpha-androstanedione or 5-alpha-androstanediol to increase levels of dihydrotestosterone in humans, a hormone which also offers the benefit of regulating protein synthesis and increasing skeletal muscle mass.

The use of androgenic hormones is often thought to entail some risk, as increasing the level of such hormones may also be relevant to the development of undesirable side effects such as gynecomastia, water retention (edema), unfavorable alterations in cholesterol levels (increased heart disease risk) and increased blood pressure to name just a few. If an individual is seeking solely to increase skeletal muscle mass, and is not in need of androgen replacement, then the methods regarding the use of androgen precursors may be less than ideal. It therefore became to focus of this inventor to find another distinct mechanism in the body that plays an important role in the regulation of protein synthesis, and can be affected externally by the similar use of a precursor compound to an active constituent in said mechanism to enhance the buildup of skeletal muscle tissue.

This invention relates a method of administering arachidonic acid derivatives for the purpose of increasing the level of the prostaglandin PGF2alpha and subsequently skeletal muscle mass. PGF2alpha is not an androgenic steroid, but an endogenous prostaglandin. It is referred to commonly as an inflammatory hormone, and is related to several biological functions including immunity, response to allergens, intestinal mobility and blood flow in various regions of the body. PGF2alpha is also closely tied to skeletal muscle protein synthesis in the body (Biochem J 1983 Sep. 15;214(3):1011-4), and represents an important new target for the external modulation of skeletal muscle mass distinct from the mechanisms involving male sex steroids. This method of using arachidonic acid for increasing PGF2alpha and skeletal muscle mass is an ideal solution for an individual in need of such treatment, because PGF2alpha is non-steroidal, and can increase protein synthesis and muscle mass without the potential undesirable side effects associated with altering sex steroid levels with androgen precursor hormones.

### BRIEF SUMMARY OF THE INVENTION

Prior art relates several novel methods of using precursors to hormones that regulate protein synthesis for the purpose of increasing the levels of said hormones, which ultimately can increase skeletal muscle mass. Although the suggested practice of using precursors to physiologically active hormones seems quite sound, the target hormones in the cited art, namely androgenic steroids, may be less than ideal in many cases, particularly in those where increases in skeletal muscle mass are desired but the potential side effects of androgens contraindicates their use. The problem of the present invention is therefore to provide a precursor to a target hormone that can also be used to increase skeletal muscle mass when administered, but is completely non-steroidal. According to the invention this problem is solved by the oral use of an arachidonic acid derivative, a direct precursor to the prostaglandin PGF2alpha. This method is ideal because it is natural, non-toxic, quickly metabolized to active form after oral administration, and can increase skeletal muscle mass without the potential side effects of androgenic precursors.

### DETAILED DESCRIPTION OF THE INVENTION

Arachidonic acid is a naturally occurring polyunsaturated fat, belonging to the Omega-6 family of fatty acids. It is considered an essential fatty acid (EFA), because it is an essential nutrient that your body can't produce itself. The only way you can get arachidonic acid is through the food you eat. It is obtained in small amounts in the average human diet, coming from various plant and animal sources including milk. Arachidonic acid has furthermore been identified as a vital precursor to numerous hormones in the body including prostaglandins, prostacyclin (PGI2), leukotrienes, and thromboxanes.

Studies by have fundamentally proven the in-vitro conversion of arachidonic acid to the prostaglandin PGF2alpha. Experiments by Berlin et al. (Acta Physiol Scand 1979 August;106(4):441-5) used 14C-labeled arachidonic acid to chart the metabolism of this essential fatty acid into various prostaglandins in human skeletal muscle and kidney homogenates. Those prostaglandins produced during this incubation include PGD2, PGE2, PGF2 alpha and 6-keto-PGF1 alpha. Further studies with labeled arachidonic acid have fundamentally proven the in-vivo conversion of this fatty acid into PGF2alpha (Acta Physiol Scand 1979 July;106(3):307-12). In this investigation the labeled metabolites of arachidonic acid were measured in serum extracted from the forearm and kidney of human volunteers after direct infusion into the brachial or renal artery. PGD2, PGE2, PGF2 alpha, 6-keto-PGF1 alpha and 13,14-dihydro-15-keto-PGE2 (Me) were all found in this experiment.

The prostaglandin PGF2alpha has also been proven to play a vital role in skeletal muscle protein synthesis. In fact, it is one of the prostaglandins most closely tied to protein synthesis, and therefore the primary focus of this invention. Studies conducted by Smith et al. (Biochem J 1983 Jul. 15;214(1):153-61) have fundamentally proven the importance of PGF2alpha in stimulating protein synthesis in-vitro, by testing the effects of various arachidonic acid metabolites when incubated with intact rabbit muscle that was intermittently placed under stretch stimulus. In this study two prostaglandins, F2 alpha and A1, increased rates of protein synthesis in unstimulated muscles, but prostaglandins E2 and D2 and the leukotrienes C4 and D4 failed to do so. Further studies with the cox-1 enzyme inhibitors ibuprofen and acetaminophen, which exhibit their anti-inflammatory actions by inhibiting the synthesis of prostaglandins, suggest that these drugs can profoundly diminish the anabolic response of muscle to resistance exercise by inhibiting the normal post-exercise increase in levels of PGF2alpha (Clin Endocrinol Metab 2001 October;86(10):5067-7). A search of the prior art does not reveal any investigations into what effect additional arachidonic acid in the diet would have on total protein synthesis or skeletal muscle mass.

Prior art also does not disclose any investigations regarding the effect oral arachidonic acid would have on the serum level of PGF2alpha. Human tests carried out by Kelley et al. (Lipids 1998 February;33(2):125-30), however, did look at the effect of oral arachidonic acid on in-vitro immune response as measured by the secretion of different prostaglandins and immune system factors. In this study, the in-vitro secretion of LTB4 and PGE2, as demonstrated by Influenza antibody titers determined on drawn blood, did seem to measurably increase after oral administration of 200 mg and 1.5 g of supplemented arachidonic acid per day. This suggested to this inventor that a similar increase might be noted in-vivo with other prostaglandins not measured in this experiment including PGF2alpha.

After learning of the in-vitro and in-vivo conversion of arachidonic acid to PGF2alpha, plus the role PGF2alpha plays in the regulation of skeletal muscle protein synthesis, it became the focus of this invention that skeletal muscle mass can be increased by the oral administration of arachidonic acid. In an effort to prove this theory a clinical study was therefore undertaken by the inventor. Specifically, it was the intention of this inventor to prove that arachidonic acid would act as an effective in-vivo peroral PGF2alpha precursor in man capable of raising and sustaining elevated PGF2alpha levels, and that the resultant increases in levels of PGF2alpha would result in increases in the level of skeletal muscle mass.

An effective oral daily dosage of arachidonic acid is between 100 mg to 5,500 mg. It is ideally provided as a soft gelatin capsule or oral liquid, due to the fact that arachidonic acid is in the form of free flowing oil at room temperature. Due to the rapidity in which the discussed compound is metabolized in the body, the total daily dosage can be further subdivided for a more sustained blood hormone concentration, with 2-3 applications per day being most preferred.

Although the free acid or triglyceride form of arachidonic acid is preferred for oral administration, there are other derivatives of arachidonic acid that can be used to similar benefit. These derivatives have an effective oral dosage of between 100 mg and 5,500 mg. This includes ethyl and methyl arachidonate, which are arachidonic acid molecules modified with ethyl and methyl ester, respectively. Ethyl esters of fatty acids are more common in dietary supplements, and are even used in vitamins such as Vitamin E. Although ethyl esters of fatty acids have been shown in studies to be absorbed at an inferior level to triglycerides (Arzneimittelforschung. 1990 June;40(6):700-4), they can be used in higher doses in order to reap the same benefits as the base triglyceride. Ethyl arachidonate is a therapeutic equivalent to the triglyceride arachidonic acid, except that a larger dose will be required for the same benefit on muscle growth. For the purposes of this invention, a ratio of 1.5:1 works most effectively. A daily dosage of 1,000 mg of arachidonic acid triglyceride is equivalent to approximately 1,500 mg of ethyl arachidonate, for the purpose of increasing PGF2alpha levels and muscle growth. Arachidonic acid can be modified with the addition of different salts, including sodium or arginine. Evidence with another fatty acid, eicosapentaenoic acid, suggests that its arginine salt (eicosapentaenoic acid arginine) is better absorbed than the ethyl ester of the same (Lipids. 1987 October;22(10):711-4). These salts offer advantages in regards to manufacture, processing and encapsulation, and are therefore of interest.

The present invention also provides methods of using pharmaceutical compositions of the inventive arachidonic acid derivative compounds. Such pharmaceutical compositions may be for administration for injection, or for oral, nasal, transdermal or other forms of administration, including, e.g., by intravenous, intradermal, intramuscular, intramammary, intraperitoneal, intrathecal, intraocular, retrobulbar, intrapulmonary (e.g., aerosolized drugs) or subcutaneous injection (including depot administration for long term release); by sublingual, anal, vaginal, or by surgical implantation, e.g., embedded under the splenic capsule, brain, or in the cornea. The treatment may consist of a single dose or a plurality of doses over a period of time. In general, comprehended by the invention are pharmaceutical compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 90), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. The pharmaceutical compositions optionally may include still other pharmaceutically acceptable liquid, semisolid, or solid diluents that serve as pharmaceutical vehicles, excipients, or media, including but are not limited to, polyoxyethylene sorbitan monolaurate, magnesium stearate, methyl- and propylhydroxybenzoate, starches, sucrose, dextrose, gum acacia, calcium phosphate, mineral oil, cocoa butter, and oil of theobroma. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form. Implantable sustained release formulations are also contemplated, as are transdermal formulations.

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets, and powder that may or may not be dissolved in a liquid. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K., Modern Pharmaceutics, Edited by G. S. Banker and C. T. Rhodes Chapter 10, 1979, herein incorporated by reference. In general, the formulation will include the inventive compound, and inert ingredients which allow for protection against the stomach environment, and release of the biologically active material in the intestine.

Also specifically contemplated are oral dosage forms of the above inventive compounds. If necessary, the compounds may be chemically modified so that oral delivery is efficacious. Generally, the chemical modification contemplated is the attachment of at least one moiety to the compound molecule itself, where said moiety permits (a) inhibition of proteolysis; and (b) uptake into the blood stream from the stomach or intestine. Also desired is the increase in overall stability of the compound and increase in circulation time in the body. Examples of such moieties include: Polyethylene glycol, copolymers of ethylene glycol and propylene glycol, carboxymethyl cellulose, dextran, polyvinyl alcohol, polyvinyl pyrrolidone and polyproline (Abuchowski and Davis, Soluble Polymer-Enzyme Adducts, Enzymes as Drugs, Hocenberg and Roberts, eds., Wiley-Interscience, New York, N.Y., (1981), pp 367-383; Newmark, et al., J. Appl. Biochem. 4:185-189 (1982)). Other polymers that could be used are poly-1,3-dioxolane and poly-1,3,6-tioxocane. Preferred for pharmaceutical usage, as indicated above, are polyethylene glycol moieties.

For the oral delivery dosage forms, it is also possible to use a salt of a modified aliphatic amino acid, such as sodium N-(8-[2-hydroxybenzoyl]amino)caprylate (SNAC), as a carrier to enhance absorption of the therapeutic compounds of this invention. The clinical efficacy of a heparin formulation using SNAC has been demonstrated in a Phase II trial conducted by Emisphere Technologies. See U.S. Pat. No. 5,792,451, “Oral drug delivery composition and methods”.

The therapeutic can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, .alpha.-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

Controlled release formulation may be desirable. The drug could be incorporated into an inert matrix which permits release by either diffusion or leaching mechanisms e.g., gums. Slowly degenerating matrices may also be incorporated into the formulation, e.g., alginates, polysaccharides. Another form of a controlled release of this therapeutic is by a method, wherein the drug is enclosed in a semipermeable membrane which allows water to enter and push drug out through a single small opening due to osmotic effects. Some enteric coatings also have a delayed release effect.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for increasing muscle mass, which method comprises administering orally to a human in need of such treatment an amount of an arachidonic acid derivative which is effective in raising the level of the endogenous prostaglandin PGF2 alpha.

2. The method of claim 1, where the arachidonic acid derivative is triglyceride arachidonic acid.

3. The method of claim 1, where the arachidonic acid derivative is ethyl arachidonate.

4. The method of claim 1, where the arachidonic acid derivative is methyl arachidonate.

5. The method of claim 1, where the arachidonic acid derivative is arachidonic acid sodium.

6. The method of claim 1, where the arachidonic acid derivative is arachidonic acid arginine.

7. The method of claim 1, wherein the arachidonic acid derivative is in an oral pharmaceutical composition selected from the group consisting of powder, tablets, capsules, pills, troches, lozenges, cachets and pellets.
